(19) United States
(12) Patent Application Publication
(10) Pub. No.: US 2009/0123983 A1
(43) Pub. Date: May 14, 2009

(54) Processes for preparing an intermediate of sitagliptin via enzymatic reduction

(76) Inventor: Valerie Niddam-Hildesheim, Kadima (IL)

Correspondence Address: KENYON & KENYON LLP, ONE BROADWAY, NEW YORK, NY, 10004, US

(21) Appl. No.: 12/286,936

(22) Filed: Oct. 3, 2008

Related U.S. Application Data
(60) Provisional application No. 60/977,210, filed on Oct. 3, 2007.

(51) Int. Cl. C12P 7/62; C07C 69/732

(52) U.S. Cl. 435/135; 560/53

(57) ABSTRACT

The invention provides enzymatic reduction processes for the preparation of 4-(2,4,5-trifluorophenyl)-3-hydroxybutanoate, particularly, (S)-methyl 4-(2,4,5-trifluorophenyl)-3-hydroxybutanoate, a key intermediate in the synthesis of Sitagliptin, and the (S)- and (R)-enantiomers of methyl 4-(2,4,5-trifluorophenyl)-3-hydroxybutanoate in high enantiomeric purity.

### CROSS-REFERENCE TO RELATED APPLICATIONS

The present invention claims the benefit of the following U.S. Provisional Patent Application No. 60/977,210, filed Oct. 3, 2007. The contents of this application is incorporated herein by reference.

### FIELD OF THE INVENTION

The invention is directed to enzymatic reduction processes for the preparation of methyl 4-(2,4,5-trifluorophenyl)-3-hydroxybutanoate. In particular, the invention is directed to enzymatic reduction processes for the preparation of (S)-methyl 4-(2,4,5-trifluorophenyl)-3-hydroxybutanoate, a key intermediate in the synthesis of Sitagliptin.

### BACKGROUND OF THE INVENTION

Sitagliptin phosphate, 3(R)-amino-1-(3-(trifluoromethyl)-5,6,7,8-tetrahydro-(1,2,4)triazolo(4,3-a)pyrazin-7-yl)-4-(2,4,5-trifluorophenyl)butan-1-one phosphate, of Formula-1 has the following chemical structure:

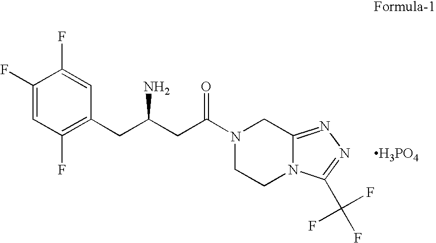

Sitagliptin phosphate is a glucagon-like peptide 1 metabolism modulator, hypoglycemic agent, and dipeptidyl peptidase IV inhibitor. Sitagliptin phosphate is currently marketed in the United States under the tradename JANUVIA™ in its monohydrate form. JANUVIA™ is indicated to improve glycemic control in patients with type 2 diabetes mellitus.

PCT Publication No. WO 2004/087650 (“WO '650”) refers to the synthesis of sitagliptin via the stereoselective reduction of methyl 4-(2,4,5-trifluorophenyl)-3-oxobutanoate to produce the sitagliptin intermediate (S)-methyl 4-(2,4,5-trifluorophenyl)-3-hydroxybutanoate. WO '650, p. 19, example 2. Example 2 of WO '650 discloses that the stereoselective reduction is performed by hydrogenation with H₂ and (S)-BINAP-RuCl₂ catalyst in the presence of hydrochloric acid. The process is illustrated in Scheme 1 below.

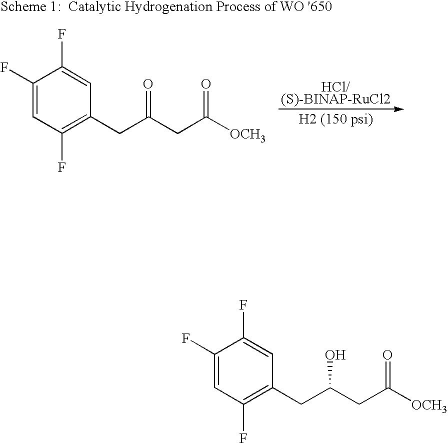

PCT Publication No. WO 2004/085661 (“WO '661”) refers to the synthesis of sitagliptin via the stereoselective reduction of a substituted enamine with PtO₂. WO '661, pp. 13-18 (example 1, scheme 2). PCT Publication No. WO 2004/085378 (“WO '378”) refers to the synthesis of sitagliptin via the stereoselective reduction of an enamine with [Rh(cod)Cl]2 and (R,S) t-butyl Josiphos (a ferrocenyl diphosphine ligand). WO '378, example 1 (scheme 2).

### SUMMARY OF THE INVENTION

In one embodiment, the present invention provides a process for preparing (S) or (R) methyl 4-(2,4,5-trifluorophenyl)-3-hydroxybutanoate, comprising:

a) combining methyl 4-(2,4,5-trifluorophenyl)-3-oxobutanoate of formula:

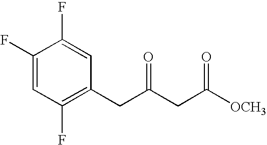

an enzyme that stereoselectively reduces a ketone to form an alcohol, and a co-factor, to obtain a reaction mixture;

b) maintaining the mixture to obtain (S) or (R) methyl 4-(2,4,5-trifluorophenyl)-3-hydroxybutanoate.

In one embodiment, the present invention provides a stereoselective enzymatic reduction processes for the preparation of 4-(2,4,5-trifluorophenyl)-3-hydroxybutanoate, particularly, (S)-methyl 4-(2,4,5-trifluorophenyl)-3-hydroxybutanoate, a key intermediate in the synthesis of Sitagliptin, and the (S)- and (R)-enantiomers of methyl 4-(2,4,5-trifluorophenyl)-3-hydroxybutanoate in high enantiomeric purity.

In one embodiment, the invention provides (S)-methyl 4-(2,4,5-trifluorophenyl)-3-hydroxybutanoate having an enantiomeric purity greater than about 87 percent, preferably, greater than about 95 percent, and, more preferably, greater than about 98 percent, as determined by HPLC.

In one embodiment, the invention further provides (R)-methyl 4-(2,4,5-trifluorophenyl)-3-hydroxybutanoate having an enantiomeric purity greater than about 86 percent, preferably, greater than about 95 percent, and, more preferably, greater than about 99 percent, as determined by HPLC.

In one embodiment, present invention provides a process for preparing methyl 4-(2,4,5-trifluorophenyl)-3-hydroxybutanoate comprises forming a solution comprising methyl 4-(2,4,5-trifluorophenyl)-3-oxobutanoate, a ketoreductase enzyme selected from the group consisting of KRED-NADH-121, KRED-NADH-124, KRED-NADH-128, KRED-NADH-108, KRED-NADH-110, KRED-NADH-116, KRED-NADH-122, KRED-NADH-125, KRED-140, KRED-137, KRED-NADH-112, KRED-NADH-114, KRED-NADH-117, KRED-NADH-123, KRED-NADH-126 and KRED-NADH-129, and a co-factor, and maintaining the solution, preferably with stirring, for a time sufficient to convert 4-(2,4,5-trifluorophenyl)-3-oxobutanoate to methyl 4-(2,4,5-trifluorophenyl)-3-hydroxybutanoate by enzymatic reduction. The enzymes are available from Codexis, Redwood City, Calif.

In one embodiment, present invention provides the (R)-enantiomer of methyl 4-(2,4,5-trifluorophenyl)-3-hydroxybutanoate, (S)-methyl 4-(2,4,5-trifluorophenyl)-3-hydroxybutanoate, is predominately formed when the enzyme is selected from the group consisting of KRED-NADH-112, KRED-NADH-114, KRED-NADH-117, KRED-NADH-123, KRED-NADH-126 and KRED-NADH-129.

In one embodiment, present invention provides the (S)-enantiomer of methyl 4-(2,4,5-trifluorophenyl)-3-hydroxybutanoate, (R)-methyl 4-(2,4,5-trifluorophenyl)-3-hydroxybutanoate, a key Sitagliptin intermediate, is predominately formed when the enzyme is selected from the group consisting of KRED-NADH-121, KRED-NADH-124, KRED-NADH-128, KRED-NADH-108, KRED-NADH-110, KRED-NADH-116, KRED-NADH-122, KRED-NADH-125, KRED-140, and KRED-137.

In one embodiment, present invention provides the invention further provides a process for preparing Sitagliptin. The process comprises preparing (S)-methyl 4-(2, 4,5-trifluorophenyl)-3-hydroxybutanoate with the enzymatic reduction process of the invention, and converting the (S)-methyl 4-(2,4,5-trifluorophenyl)-3-hydroxybutanoate into Sitagliptin.

### DETAILED DESCRIPTION OF THE INVENTION

As used herein, the term “ketoreductase,” “ketoreductase enzyme,” or “KRED” refers to an enzyme that catalyzes the reduction of a ketone to form the corresponding alcohol in a stereoselective manner, optionally with the aid of co-factor. Ketoreductase enzymes include, for example, those classified under the EC numbers of 1.1.1. Such enzymes are given various names in addition to ketoreductase, including, but not limited to, alcohol dehydrogenase, carbonyl reductase, lactate dehydrogenase, hydroxyacid dehydrogenase, hydroxyisocaproate dehydrogenase, β-hydroxybutyrate dehydrogenase, steroid dehydrogenase, sorbitol dehydrogenase, and aldoreductase. NADPH-dependent ketoreductases are classified under the EC number of 1.1.1.2 and the CAS number of 9028-12-0. NADH-dependent ketoreductases are classified under the EC number of 1.1.1.1 and the CAS number of 9031-72-5. Ketoreductases are commercially available, for example, from Codexis, Inc. under the catalog numbers KRED-101 to KRED-177.

The KRED can be a wild-type or a variant enzyme. Sequences of wild type and variant KRED enzymes are provided in WO2005/017135, incorporated herein by reference.

KRED enzymes are commercially available. Examples of these include KRED-NADH-121, KRED-NADH-124, KRED-NADH-128, KRED-NADH-108, KRED-NADH-110, KRED-NADH-116, KRED-NADH-122, KRED-NADH-125, KRED-140, KRED-137, KRED-NADH-112, KRED-NADH-114, KRED-NADH-117, KRED-NADH-123, KRED-NADH-126, and KRED-NADH-129. The KRED enzymes used are preferably selected from the group consisting of at least one of the predominant enzyme in each of: KRED-NADH-121, KRED-NADH-124, KRED-NADH-128, KRED-NADH-108, KRED-NADH-110, KRED-NADH-116, KRED-NADH-122, KRED-NADH-125, KRED-140, KRED-137, and combinations thereof. Preferably the enzyme is KRED-NADH-108, KRED-NADH-110, KRED-NADH-116, and KRED-NADH-12. Most preferably the enzyme is KRED-NADH-108, KRED-NADH-110.

Preferably, the ketoreductase is isolated. The ketoreductase can be separated from any host, such as mammals, filamentous fungi, yeasts, and bacteria. The isolation, purification, and characterization of a NADH-dependent ketoreductase is described in, for example, in Kosjek et al., Purification and Characterization of a Chemotolerant Alcohol Dehydrogenase Applicable to Coupled Redox Reactions, Biotechnology and Bioengineering, 86:55-62 (2004). Preferably, the ketoreductase is synthesized. The ketoreductase can be synthesized chemically or using recombinant means. The chemical and recombinant production of ketoreductases is described in, for example, in European patent no. EP 0918090. Preferably, the ketoreductase is synthesized using recombinant means in Escherichia coli. Preferably, the ketoreductase is purified, preferably with a purity of about 90% or more, more preferably with a purity of about 95% or more. Preferably, the ketoreductase is substantially cell-free.

As used herein, the term “co-factor” refers to an organic compound that operates in combination with an enzyme which catalyzes the reaction of interest. Co-factors include, for example, nicotinamide co-factors such as nicotinamide adenine dinucleotide (“NAD”), reduced nicotinamide adenine dinucleotide (“NADH”), nicotinamide adenine dinucleotide phosphate (“NADP⁺”), reduced nicotinamide adenine dinucleotide phosphate (“NADPH”), and any derivatives or analogs thereof.

The invention is directed to processes for the preparation of methyl 4-(2,4,5-trifluorophenyl)-3-hydroxybutanoate, particularly, the Sitagliptin intermediate (S)-methyl 4-(2,4,5-trifluorophenyl)-3-hydroxybutanoate, via enzymatic reduction of methyl 4-(2,4,5-trifluorophenyl)-3-oxobutanoate comprising an enzymatic reduction of methyl 4-(2,4,5-trifluorophenyl)-3-oxobutanoate for the preparation of (S)-methyl 4-(2,4,5-trifluorophenyl)-3-hydroxybutanoate of the invention, and (S)- and (R)-methyl 4-(2,4,5-trifluorophenyl)-3-hydroxybutanoate of high enantiomeric purity.

Methyl 4-(2,4,5-trifluorophenyl)-3-oxobutanoate can be prepared according to any method known in the art, for example, according to the procedure disclosed in PCT Publication No. WO 2004/087650, which comprises reacting oxalyl chloride with 2,5-difluorophenylacetic acid in the presence of dichloromethane, followed by refluxing the obtained Meldrum's acid in methanol to obtain the desired product.

The enzymatic reduction processes of the invention in which the enzyme acts as a reduction catalyst are environmentally advantageous compared to the use of metal catalysts in the prior art. The use of the enzymes is also typically lower in cost than the ruthenium catalyst used in WO 2004/087650. In addition, metal catalysts, such as the ruthenium catalyst used in WO 2004/087650, the platinum catalyst disclosed in WO 2004/085661, and the rhodium catalyst discloses in WO 2004/085378, can leave trace amounts of the metal in the final product, and, thus, are problematic for the manufacture of pharmaceutical products.

The invention provides (S)-methyl 4-(2,4,5-trifluorophenyl)-3-hydroxybutanoate of the following formula

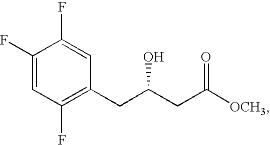

having an enantiomeric purity greater than about 86 percent, preferably, greater than about 95 percent, and, more preferably, greater than about 99 percent, as determined by HPLC.

The invention further provides (R)-methyl 4-(2,4,5-trifluorophenyl)-3-hydroxybutanoate of the following formula

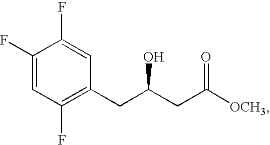

having an enantiomeric purity greater than about 87 percent, preferably, greater than about 95 percent, and, more preferably, greater than about 98 percent, as determined by HPLC.

The process of the invention for the preparation of the Sitagliptin intermediate (S)-methyl 4-(2,4,5-trifluorophenyl)-3-hydroxybutanoate of the formula

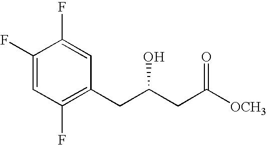

comprises combining methyl 4-(2,4,5-trifluorophenyl)-3-oxobutanoate of the formula

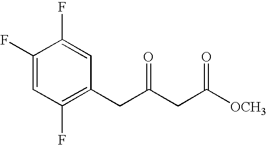

with an enzyme and a co-factor that catalyze a ketone to an alcohol in a stereoselective manner to obtain a reaction mixture, and maintaining the reaction mixture to obtain the intermediate. Preferably the enzyme is a ketoreductase (KRED). The enzyme can be isolated from a natural source or synthesized with recombinant technology.

Preferably, the ketoreductase is one that is capable of producing (S)- or (R)-methyl 4-(2,4,5-trifluorophenyl)-3-hydroxybutanoate with a d.e. of about 90% or higher in the processes of the invention. Preferably, the ketoreductase is one that capable of producing (S)- or (R)-methyl 4-(2,4,5-trifluorophenyl)-3-hydroxybutanoate with a yield of about 50% or higher in the processes of the invention.

Preferably, the ketoreductase is selected from the group consisting of NADH-dependent ketoreductases and NADPH-dependent ketoreductases. Suitable ketoreductases include, but are not limited to, Codexis Inc's products with catalog numbers KRED-NADH-121, KRED-NADH-124, KRED-NADH-128, KRED-NADH-108, KRED-NADH-110, KRED-NADH-116, KRED-NADH-122, KRED-NADH-125, KRED-140, KRED-137, KRED-NADH-112, KRED-NADH-114, KRED-NADH-117, KRED-NADH-123, KRED-NADH-126, KRED-NADH-129, and combinations thereof. Preferably, the ketoreductase is selected from the group consisting of the predominant enzyme in each of Codexis Inc's products with catalog numbers KRED-NADH-121, KRED-NADH-124, KRED-NADH-128, KRED-NADH-108, KRED-NADH-110, KRED-NADH-116, KRED-NADH-122, KRED-NADH-125, KRED-140, KRED-137, and combinations thereof. Preferably the enzyme is KRED-NADH-108, KRED-NADH-110, KRED-NADH-116, and KRED-NADH-12. More preferably, the ketoreductase is selected from the group consisting of KRED-NADH-108, KRED-NADH-110, and combinations thereof.

The co-factor is selected from the group consisting of NADH, NADPH, NAD⁺, NADP⁺, salts thereof, and mixtures thereof. Preferably, when the ketoreductase is NADH-dependent, the co-factor is selected from the group consisting of NADH, NAD⁺, salts thereof, and mixtures thereof. More preferably, the co-factor is NADH or a salt thereof. Preferably, when the ketoreductase is NADPH-dependent, the co-factor is selected from the group consisting of NADPH, NADP⁺, salts thereof, and mixtures thereof. More preferably, the co-factor is NADPH or a salt thereof. Examples of salts of the co-factors include NAD tetra(cyclohexyl ammonium) salt, NAD tetrasodium salt, NAD tetrasodium hydrate, NADP⁺ phosphate hydrate, NADP⁺ phosphate sodium salt, and NADH dipotassium salt.

In one embodiment, the process of the invention is carried out in a buffer. Preferably, the buffer has a pH of from about 4 to about 9, more preferably from about 4 to about 8, more preferably from about 5 to about 8, most preferably from about 6 to about 8 or about 5 to about 7. Preferably, the buffer is a solution of a salt. Preferably, the salt is selected from the group consisting of potassium phosphate, magnesium sulfate, and mixtures thereof. Optionally, the buffer comprises a thiol. Preferably, the thiol is DTT. Preferably, the thiol reduces the disulfide bond in the enzyme.

In one embodiment, the process of the invention is carried out at a temperature of about 10° C. to about 50° C. Preferably, the process is carried out at room temperature, at a temperature of about 20° C. to about 30° C., or at about 25° C. to about 35° C. Preferably, the process is carried out at a temperature of about 25° C. to about 30° C., such as at a temperature of about 30° C.

Optionally, the reaction mixture further comprises a co-factor regeneration system. A co-factor regeneration system comprises a substrate and a dehydrogenase. The reaction between the substrate and dehydrogenase enzyme regenerates the co-factor. Preferably, the co-factor regeneration system comprises a substrate/dehydrogenase pair selected from the group consisting of D-glucose/glucose dehydrogenase, sodium formate/formate dehydrogenase, phosphite/phosphite dehydrogenase, and isopropanol and ketoreductase/hydrogenase. Preferably, the glucose dehydrogenase is selected from the group consisting of the predominant enzyme in each of Codexis Inc's products with catalog numbers GDH-102, GDH-103, GDH-104, and mixtures thereof. Preferably, the glucose dehydrogenase is the enzyme in GDH-104. Preferably, the formate dehydrogenase is the predominant enzyme in Codexis Inc's product with catalog number FDH-101. Preferably, the phosphite dehydrogenase is the predominant enzyme in Codexis Inc's product with catalog number PDH-101.

In one embodiment, the process of the invention is carried out in the presence of a solvent, such as an organic solvent. Preferably, the organic solvent is water-miscible, such as water-miscible alcohols, acetonitrile, tetrahydrofuran, and dimethylsulfoxide. Preferably, the alcohol is a C₁-C₄ alcohol, more preferably methanol or IPA (iso-propyl alcohol). With a water miscible solvent, particularly alcohols and dimethylsulfoxide, the reaction medium is mostly water, which makes the reaction more environmentally friendly.

The process can comprise the following steps: (a) dissolving methyl 4-(2,4,5-trifluorophenyl)-3-oxobutanoate in a solvent; and (b) combining the solution from (a) with a buffer containing a co-factor and a ketoreductase. Optionally, the solution comprises a co-factor regeneration system. Preferably, the obtained mixture is maintained for a period of time sufficient to obtain (S)-methyl 4-(2,4,5-trifluorophenyl)-3-hydroxybutanoate. Preferably, the reaction is maintained at a temperature of about 10° C. to about 50° C. or about 20° C. to about 40° C., more preferably at a temperature of about 25° C. to about 30° C., or about 30° C. Preferably, the reaction is maintained for about 0.5 hours or more, about 1.5 hours or more, or about 2.5 hours or more. Preferably, the reaction is maintained for about 50 hours or less. Preferably, the reaction is maintained for about 3 hours to about 40 hours, more preferably for about 6 hours to about 24 hours or about 6 hours to about 16 hours. The reaction can be stirred.

Optionally, a water-immiscible organic solvent is added to the reaction mixture, preferably after the stirring. Optionally, after the water-immiscible organic solvent is added, the reaction mixture is separated into an organic phase and an aqueous phase. Optionally, (S)-methyl 4-(2,4,5-trifluorophenyl)-3-hydroxybutanoate is recovered by evaporating the organic phase. Examples of water-immiscible organic solvents include, but are not limited to, C₂-C₈ ethers, C₃-C₈ esters such as EtOAc, C₄-C₈ ketones such as MIBK, and halogenated hydrocarbons such as DCM. Preferably, the water-immiscible organic solvent is selected from the group consisting of EtOAc, MTBE, diethyl ether, and mixtures thereof. Preferably, the water-immiscible organic solvent is EtOAc.

Optionally, the reaction mixture, preferably after the stirring, is filtered to recover the solid product, which may optionally be further purified to obtain (S)-methyl 4-(2,4,5-trifluorophenyl)-3-hydroxybutanoate.

Optionally, after the product is separated, the aqueous phase may be treated to recycle the enzyme, co-factor, and/or the dehydrogenase in the co-factor regeneration system. Optionally, the pH of the aqueous phase may be adjusted to obtain the desired pH. Optionally, the aqueous phase is evaporated to remove organic solvent residue. Optionally, the aqueous phase is reused in the process of the invention.

The corresponding (R)-enantiomer of methyl 4-(2,4,5-trifluorophenyl)-3-hydroxybutanoate, (R)-methyl 4-(2,4,5-trifluorophenyl)-3-hydroxybutanoate of the formula

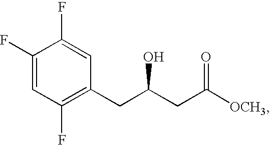

may be obtained with the process of the invention by preparing a solution comprising the methyl 4-(2,4,5-trifluorophenyl)-3-oxobutanoate and an enzyme selected from the group consisting of KRED-NADH-121, KRED-NADH-124, KRED-NADH-128, KRED-NADH-108, KRED-NADH-110, KRED-NADH-116, KRED-NADH-122, KRED-NADH-125, KRED-140, and KRED-137, and maintaining the solution, preferably with stirring, for a time sufficient to convert the 4-(2,4,5-trifluorophenyl)-3-oxobutanoate to (R)-methyl 4-(2,4,5-trifluorophenyl)-3-hydroxybutanoate by enzymatic reduction. Preferably, the enzyme is KRED-NADH-114 or KRED-NADH-117.

The invention further provides a process for preparing Sitagliptin, comprising preparing (S)-methyl 4-(2,4,5-trifluorophenyl)-3-hydroxybutanoate with an enzymatic reduction process of the invention, and converting the (S)-methyl 4-(2,4,5-trifluorophenyl)-3-hydroxybutanoate into Sitagliptin. The (S)-methyl 4-(2,4,5-trifluorophenyl)-3-hydroxybutanoate may be converted into Sitagliptin by any method known in the art; for example, by the method referred to in WO 2004/087650, hereby incorporated by reference.

Preferably, high performance liquid chromatography (“HPLC”) methods are used to determine the chemical purity of the methyl 4-(2,4,5-trifluorophenyl)-3-hydroxybutanoate. The HPLC method may comprise analyzing a sample of methyl 4-(2,4,5-trifluorophenyl)-3-hydroxybutanoate by HPLC under the following conditions:
Column: Discovery, 150 mm×4.6 mm (Supelco);
Eluent: Eluent A (0.08 percent (0.8 ml) TFA in acetonitrile (11)); Eluent B (0.1 percent (1.0 ml) TFA in water (11));
Gradient of Eluent:

| Time (min) | Eluent A (%) | Eluent B (%) |
|---|---|---|
| 0 | 40 | 60 |
| 10 | 40 | 60 |
| 30 | 70 | 30 |

Flow rate: 1.0 ml/min;
Column Temp.: 30° C.;
Detector: PDA at 210 nm.

The HPLC method for determining the enantiomeric purity of methyl 4-(2,4,5-trifluorophenyl)-3-hydroxybutanoate may also comprise analyzing a sample of methyl 4-(2,4,5-trifluorophenyl)-3-hydroxybutanoate by HPLC under the following conditions:
Column: Chiralpak-AD-H, 5 μm, 150 mm×4.6 mm;
Eluent: 5 percent 2-Propanol/95 percent n-Hexane (v/v);
Flow rate: 1.0 ml/min;
Column Temp.: 35° C.
Detector: PDA/UV at 260 nm.

Having described the invention with reference to certain preferred embodiments, other embodiments will become apparent to one skilled in the art from consideration of the specification. The invention is further defined by reference to the following examples. It will be apparent to those skilled in the art that many modifications, both to materials and methods, may be practiced without departing from the scope of the invention.

### EXAMPLES

### HPLC Analysis

(a) Chemical Purity

Samples of methyl 4-(2,4,5-trifluorophenyl)-3-hydroxybutanoate were analyzed by HPLC using a Discovery, 150 mm×4.6 mm (Supelco) column equipped with a photodiode array detector set at 210 nm. The column temperature was 30° C. The flow rate was 1.0 ml/minute.

Samples were gradient eluted through the column with a mixture of eluent A (0.8 ml of TFA in 11 of acetonitrile) and eluent B (1.0 ml of TFA in 11 of water). The gradient was as follows:

| Time (min) | Eluent A (%) | Eluent B (%) |
|---|---|---|
| 0 | 40 | 60 |
| 10 | 40 | 60 |
| 30 | 70 | 30 |

(b) Enantiomeric Purity

Samples of methyl 4-(2,4,5-trifluorophenyl)-3-hydroxybutanoate were analyzed by HPLC using a Chiralpak-AD-H, 5 μm, 150 mm×4.6 mm column. The column temperature was 35° C. The flow rate was 1.0 ml/minute. Samples were eluted through the column with a mixture of 5 percent by volume 2-propanol and 95 percent by volume n-hexane. The enzymes and buffer were provided by BioCatalytics Inc., Pasadena, USA, now Codexis, Redwood City, Calif.

The S-enantiomer of methyl 4-(2,4,5-trifluorophenyl)-3-hydroxybutanoate has a retention time (RT) of 10 minutes and the R-enantiomer of methyl 4-(2,4,5-trifluorophenyl)-3-hydroxybutanoate has a RT of 12 minutes under these conditions.

### Example 1

### General Procedure for Preparing (S)-methyl 4-(2,4,5-trifluorophenyl)-3-hydroxybutanoate

Recycle mixes were prepared to provide for the regeneration and recovery of the enzymes.

(a) Recycle Mix for KRED-NADH

KRED-NADH Recycle Mix A: 250 mM Potassium phosphate, 0.5 mM Dithiothreitol, 2 mM Magnesium sulfate, 1.3 mM NAD+, 80 mM D-glucose, 10 U/ml Glucose dehydrogenase, pH 7.0

KRED-NADH Recycle Mix B: 200 mM MOPS, 160 mM TRIS, 100 mM Potassium chloride, 2 mM Magnesium chloride, 1.3 mM NAD+, 80 mM D-glucose, 10 U/ml Glucose dehydrogenase, pH 7.5

(b) Recycle Mix for KRED-NADPH

KRED-NADPH Recycle Mix A: 250 mM Potassium phosphate, 0.5 mM Dithiothreitol, 2 mM Magnesium sulfate, 1.1 mM NADP+, 80 mM D-glucose, 10 U/ml Glucose dehydrogenase, pH 7.0

(c) Preparation of methyl 4-(2,4,5-trifluorophenyl)-3-hydroxybutanoate

A solution of 50 mg (250 μmol) methyl 4-(2,4,5-trifluorophenyl)-3-oxobutanoate dissolved in 0.2 ml of DMSO was added to a solution of 5 mg of the enzyme in 5 ml of recycle mix solution, and stirred for 24 hours. The product methyl 4-(2,4,5-trifluorophenyl)-3-hydroxybutanoate was extracted with 5 ml EtOAc, isolated by evaporation, and analyzed by HPLC for enantiomeric and chemical purity as described above. The results are summarized in Tables 1 and 2 below.

As shown in Table 1, the S-enantiomer of methyl 4-(2,4,5-trifluorophenyl)-3-hydroxybutanoate was obtained with an enantiomeric purity of greater than 86 percent area percent with the enzymes KRED-NADH-121, KRED-NADH-124, KRED-NADH-128, KRED-NADH-108, KRED-NADH-110, KRED-NADH-116, KRED-NADH-122, KRED-NADH-125, KRED-140, and KRED-137. As shown in Table 2, the (R)-enantiomer of methyl 4-(2,4,5-trifluorophenyl)-3-hydroxybutanoate was obtained with an optical enantiomeric purity of greater than 87 percent area percent with the enzymes KRED-NADH-112, KRED-NADH-114, KRED-NADH-117, KRED-NADH-123, KRED-NADH-126 and KRED-NADH-129.

TABLE 1

| | | | Enantiomeric Purity | | | Chemical Purity | |
|---|---|---|---|---|---|---|---|
| | | | (peak area %) | | | (peak area %) | |
| | | | Wave | | | (measured with a wave | |
| Exp. | | Recycle | Length | | | length of 210 nm) | |
| No. | Enzyme | mix | (nm) | R-enantiomer | S-enantiomer | RT~10 (min) | RT~7 (min) |
| 1 | KRED-NADH-121 | B-KRED- | 210 | 1.8 | 98.2 | 97.2 | 2.8 |
| | | NADH | | | | | |
| 2 | KRED-NADH-124 | B-KRED- | 210 | 6.9 | 93.1 | 99.1 | 0.9 |
| | | NADH | | | | | |
| 3 | KRED-NADH-128 | B-KRED- | 210 | 8.5 | 91.5 | 98.0 | 2.0 |
| | | NADH | | | | | |
| 4 | KRED-NADH-108 | A-KRED- | 260 | 0.63 | 99.37 | 0.9 | 99.1 |
| | | NADH | | | | | |
| 5 | KRED-NADH-110 | A-KRED- | 260 | 0.11 | 99.89 | 0.3 | 99.7 |
| | | NADH | | | | | |
| 6 | KRED-NADH-116 | A-KRED- | 260 | 1.9 | 98.1 | 24.5 | 75.5 |
| | | NADH | | | | | |
| 7 | KRED-NADH-122 | A-KRED- | 210 | 1.4 | 98.6 | 10.2 | 89.8 |
| | | NADH | | | | | |
| 8 | KRED-NADH-125 | A-KRED- | 210 | 7.8 | 92.2 | 84.1 | 15.9 |
| | | NADH | | | | | |
| 9 | KRED-140 | A-KRED- | 210 | 5.3 | 94.7 | 99.9 | 0.1 |
| | | NADPH | | | | | |
| 10 | KRED-137 | A-KRED- | 210 | 13.5 | 86.5 | 99.3 | 0.7 |
| | | NADPH | | | | | |

TABLE 2

| | | | | | Wave |
|---|---|---|---|---|---|
| Exp | | | Enantiomers (%) | | Length |
| No. | Enzyme | Recycle mix | R-enantiomer | S-enantiomer | (nm) |
| 1 | KRED-NADH-112 | A-KRED-NADH | 93.0 | 7.0 | 260 |
| 2 | KRED-NADH-114 | A-KRED-NADH | 95.4 | 4.6 | 260 |
| 3 | KRED-NADH-117 | A-KRED-NADH | 98.3 | 1.7 | 210 |
| 4 | KRED-NADH-123 | A-KRED-NADH | 87.5 | 12.5 | 210 |
| 5 | KRED-NADH-126 | A-KRED-NADH | 88.2 | 11.8 | 210 |
| 6 | KRED-NADH-129 | A-KRED-NADH | 89.9 | 10.1 | 210 |

TABLE 3
Lot Nos., and years of production:

| Product Number | Lot Number | Production Year |
|---|---|---|
| KRED-NADH-108 | 171706CL | 2006 |
| KRED-NADH-110 | 073106CL | 2006 |
| KRED-NADH-112 | 140207PB | 2007 |
| KRED-NADH-114 | 211006WW | 2006 |
| KRED-NADH-116 | 100906CL | 2006 |
| KRED-NADH-117 | 082707WW | 2007 |
| KRED-NADH-121 | 122206WW | 2006 |
| KRED-NADH-122 | 121806WW | 2006 |
| KRED-NADH-123 | 222006WW | 2006 |
| KRED-NADH-124 | 013107MM | 2007 |
| KRED-NADH-125 | 191806CL | 2006 |
| KRED-NADH-126 | 013107WW | 2007 |
| KRED-NADH-128 | 213107WW | 2007 |
| KRED-NADH-129 | 022607WW | 2007 |
| KRED-137 | 213006MM | 2006 |
| KRED-140 | 220905CL | 2005 |

What is claimed:

1. A process for preparing (S) or (R) methyl 4-(2,4,5-trifluorophenyl)-3-hydroxybutanoate, comprising:
a) combining methyl 4-(2,4,5-trifluorophenyl)-3-oxobutanoate of formula: 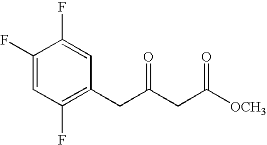
an enzyme that stereoselectively reduces a ketone to form an alcohol, and a co-factor, to obtain a reaction mixture;
b) maintaining the mixture to obtain (S) or (R) methyl 4-(2,4,5-trifluorophenyl)-3-hydroxybutanoate.

2. The process of claim 1, wherein the enzyme is a ketoreductase.

3. The process of claim 1, wherein (S) methyl 4-(2,4,5-trifluorophenyl)-3-hydroxybutanoate is obtained.

4. The process of claim 3, wherein the enzyme is selected from the group consisting of at least one of KRED-NADH-121, KRED-NADH-124, KRED-NADH-128, KRED-NADH-108, KRED-NADH-110, KRED-NADH-116, KRED-NADH-122, KRED-NADH-125, KRED-140, KRED-137, and combinations thereof.

5. The process according to claim 4, wherein the enzyme is KRED-NADH-108 or KRED-NADH-110.

6. The process of claim 1, wherein (R) methyl 4-(2,4,5-trifluorophenyl)-3-hydroxybutanoate is obtained.

7. The process of claim 6, wherein the enzyme is selected from the group consisting of at least one of KRED-NADH-112, KRED-NADH-114, KRED-NADH-117, KRED-NADH-123, KRED-NADH-126, KRED-NADH-129 and combinations thereof.

8. The process according to claim 1, wherein the (S)-methyl 4-(2,4,5-trifluorophenyl)-3-hydroxybutanoate has an enantiomeric purity of greater than about 99 percent as determined by HPLC.

9. The process according to claim 1, wherein the (R)-methyl 4-(2,4,5-trifluorophenyl)-3-hydroxybutanoate has an enantiomeric purity of greater than about 98 percent as determined by HPLC.

10. The process according to claim 1, wherein the mixture is maintained for a period of from about 2 to about 24 hours.

11. The process according to claim 1, wherein the mixture is maintained for about 3 hours to about 24 hours,

12. The process according to claim 1, wherein the mixture is maintained for about 4 hours to about 16 hours.

13. The process according to claim 1, wherein the mixture is maintained at a temperature of about 10 to about 50° C.

14. The process according to claim 1, wherein the mixture is maintained at a temperature of about 20° C. to about 40° C.

15. The process according to claim 1, wherein the mixture is maintained at a temperature of about 25° C. to about 30° C.

16. The process according to claim 1, wherein the co-factor is a nicotinamide co-factor.

17. The process of claim 16, wherein the co-factor is selected from the group consisting of nicotinamide adenine dinucleotide (“NAD”), reduced nicotinamide adenine dinucleotide (“NADH”), nicotinamide adenine dinucleotide phosphate (“NADP⁺”), reduced nicotinamide adenine dinucleotide phosphate (“NADPH”), and mixtures thereof.

18. The process according to claim 1, wherein the reaction mixture further comprises a co-factor regeneration system that comprises a dehydrogenase and substrate.

19. The process of claim 18 wherein the substrate/dehydrogenase pair is selected from the group consisting of D-glucose/glucose dehydrogenase, sodium formate/formate dehydrogenase, and phosphite/phosphite dehydrogenase.

20. The process according to claim 1, wherein the process is carried out in an organic solvent.

21. The process of claim 20, wherein the solvent is a water miscible organic solvent.

22. The process of claim 20, wherein the solvent is a C₁-C₄ alcohol or dimethylsulfoxide.

23. The process according to claim 1, wherein (R)-methyl 4-(2,4,5-trifluorophenyl)-3-hydroxybutanoate is obtained with an enantiomeric purity greater than about 98 percent, as determined by HPLC.

24. The process according to claim 1, wherein (S)-methyl 4-(2,4,5-trifluorophenyl)-3-hydroxybutanoate is obtained having an enantiomeric purity greater than about 99 percent, as determined by HPLC.

25. The process according to claim 1, comprising a preliminary step of preparing the mixture of methyl 4-(2,4,5-trifluorophenyl)-3-oxobutanoate and the enzyme by mixing a solution of methyl 4-(2,4,5-trifluorophenyl)-3-oxobutanoate and a solution of the enzyme, wherein the solution of methyl 4-(2,4,5-trifluorophenyl)-3-oxobutanoate comprises an organic solvent, and the solution of the enzyme comprises a recycle mixture.

26. The process according to claim 1, wherein (R)-methyl 4-(2,4,5-trifluorophenyl)-3-hydroxybutanoate is obtained having an enantiomeric purity greater than about 98 percent, as determined by HPLC.

27. The process according to claim 1, wherein (S)-methyl 4-(2,4,5-trifluorophenyl)-3-hydroxybutanoate is obtained having an enantiomeric purity greater than about 99 percent, as determined by HPLC.

28. A process for the preparation of Sitagliptin comprising preparing (R) or (S)-methyl 4-(2,4,5-trifluorophenyl)-3-hydroxybutanoate with the process of claim 2 and converting the (R) or (S)-methyl 4-(2,4,5-trifluorophenyl)-3-hydroxybutanoate to Sitagliptin.

29. (R)-methyl 4-(2,4,5-trifluorophenyl)-3-hydroxybutanoate, having an enantiomeric purity greater than about 98 percent, as determined by HPLC.

30. (S)-methyl 4-(2,4,5-trifluorophenyl)-3-hydroxybutanoate having an enantiomeric purity greater than about 99 percent, as determined by HPLC.
